(19) United States
(12) Patent Application Publication
(10) Pub. No.: US 2017/0028138 A1
(43) Pub. Date: Feb. 2, 2017

(54) PUSH-AND-SEPARATE SAFETY SYRINGE

(71) Applicant: Ching Hsiang Wei, Taichung City (TW)

(72) Inventor: Ching Hsiang Wei, Taichung City (TW)

(21) Appl. No.: 14/811,810

(22) Filed: Jul. 28, 2015

(51) Int. Cl. A61M 5/32; A61M 5/34

(52) U.S. Cl. CPC A61M 5/321; A61M 5/344; A61M 2005/3206

(57) ABSTRACT

A safety syringe includes a barrel, a plunger, a socket, a connector and a needle. The plunger is inserted in the barrel. The connector is inserted in the socket. The needle is connected to the connector. The socket includes a conical internal face in contact with a conical external face of the connector, an annular internal rib inserted in an annular external groove of the connector, and an annular internal groove for receiving an anchoring ring of the connector, thus retaining the connector in the socket. The plunger is movable backward to draw medicine into the barrel via the needle. The plunger is movable forward to extrude the medicine from the barrel via the needle. As the plunger is in contact with the anchoring ring, a larger force can be used to push the connector and the needle further forward from the socket to detach the needle from the socket.

### BACKGROUND OF INVENTION

1. Field of Invention

The present invention relates to a safety syringe and, more particularly, to a push-and-separate safety syringe.

2. Related Prior Art

To protect a medical worker from a needle after a syringe is used, it is necessary to provide a protective mechanism for disposing of the needle. Referring to FIG. 7, U.S. Pat. No. 8,608,694 discloses a conventional self-locking self-destroying syringe including a barrel 1, a push rod 2, a rubber plug 3, a needle seat 4 and a clamping core 5. The push rod 2 is movably inserted in the barrel 1. The rubber plug 3 is located around and connected to a front end of the push rod 2. The clamping core 5 is attached to the front end of the push rod 2. A needle is connected to the needle seat 4, which is connected to a front end of the barrel 1. In operation, the push rod 2 is first moved toward the front end of the barrel 1 to expel air out of the barrel 1. Then, the push rod 2 is moved away from the front end of the barrel 1 to draw medicine into the barrel 1. To inject the medicine into a patient, the push rod 2 is moved toward the front end of the barrel 1. Accordingly, the rubber plug 3 and the clamping core 5 are moved toward the front end of the barrel 1. At the end of the injection, the clamping core 5 is inserted in and connected to the needle seat 4. Then, the push rod 2 is moved in an opposite direction so that the needle and the needle seat 4 are inserted in the barrel 1.

The assembly of the syringe must be firm. High air-tightness is required between the front end of the barrel 1 and the needle seat 4. Otherwise, the patient might suffer a blood clot caused by air injected therein through a gap between the front end of the barrel 1 and the needle seat 4. However, the firmness and the air-tightness might be too much to allow the push rod 2 and the clamping core 5 to bring the needle and the needle seat 4 in the barrel 1. Ease in the use of the push rod 2 and the clamping core 5 to bring the needle and the needle seat 4 in the barrel 1 would adversely affect necessary firmness and air-tightness. To take these two conflicting factors into consideration, almost the entire syringe has to be redesigned, and a complicated structure and a difficult process are involved. Hence, a high cost is inevitable.

Moreover, near the end of the injection, the clamping core 5 blocks the needle seat 4 and a considerable portion of the medicine is hence left in a space between the front end of the barrel 1 and the front end of the push rod 2. The medicine could be very expensive, and the injection of a sufficient amount of the medicine might be required. In such cases, any leftover of the medicine in the barrel 1 is not allowed. To avoid such leftover, a large force must be exerted on the push rod 2 to insert the clamping core 5 into the needle seat 4. However, such a large force inevitably vibrates the syringe, particularly the needle, and such vibration of the needle might cause a serious pain to the user and might even pierce a blood vein of the user.

As discussed above, at the start of the operation, the push rod 2 is moved toward the front end of the barrel 1 to expel air out of the barrel 1. However, the clamping core 5 might be inserted in the needle seat 4 by mistake, and the needles seat 4 and the needle might be inserted into the barrel 1 when push rod 2 is moved away from the front end of the barrel 1 in an unsuccessful attempt to draw the medicine into the barrel 1.

In addition, it would be a safe procedure for a medical worker to insert the needle into the barrel 1 before detaching the syringe from the patient. To insert the needle into the barrel 1, it would be better for the medical worker to use both hands to move the push rod 2 away from the front end of the barrel 1, with one hand holding the barrel 1 and the other hand moving the push rod 2. This is difficult since the medical worker has to use one hand to press a sterilized cotton ball soaked with alcohol against the patient. Hence, in practice, the medical worker uses one hand to remove the needle from the patient before managing to use both hands to insert the needle into the barrel 1. During this period in which the needle is located out of the barrel 1 after use, the medical worker or any other person nearby is exposed to danger of being hurt by the needle.

The present invention is therefore intended to obviate or at least alleviate the problems encountered in prior art.

### SUMMARY OF INVENTION

It is the primary objective of the present invention to provide a reliable, convenient, inexpensive push-and-separate safety syringe.

To achieve the foregoing objective, the push-and-separate safety syringe includes a barrel formed with a socket at a front end, a plunger movably inserted in the barrel, a connector inserted in the socket, and a needle unit connected to the connector. The socket includes a conical internal face in contact with a conical external face of the connector. The socket includes an annular internal rib inserted in an annular external groove of the connector. The socket includes an annular internal groove for receiving an anchoring ring of the connector, thus retaining the connector in the socket. The plunger is movable backward in the barrel to draw medicine into the barrel from a bottle via the needle unit and the connector. The plunger is movable forward in the barrel to extrude the medicine out of the barrel via the connector and the needle unit. As the plunger is in contact with the anchoring ring of the connector, a larger force can be used to push the connector and the needle unit further forward away from the socket of the socket. Hence, the needle unit is disengaged from the barrel after use, and a medical worker is protected from the needle.

The needle unit is pushed away from the front end of the barrel via the plunger, not like the conventional self-locking self-destroying syringe discussed in the RELATED PRIOR ART.

The push-and-separate safety syringe is operable by a single hand.

The barrel and the needle unit can be disposed separately after use.

The insertion of the connector in the socket is firm and air-tight while facilitating the disengagement of the needle unit from the barrel.

The plunger is freely movable in the barrel to expel air out of the barrel and draw the medicine into the barrel without the risk of moving the connector and the needle unit that could happen to the self-locking self-destroying syringe discussed in the RELATED PRIOR ART.

The connector cooperates with the plunger to push the medicine out of the barrel smoothly, without experiencing much resistance.

At the end of the injection of the medicine, almost no medicine is left between the connector and the plunger.

The connector is inserted in the front end of the barrel, without exposing any portion thereof to interfere with the medical worker's operation of the syringe.

The connector and the barrel involve simple structures, without necessitating redesign of the plunger and the needle unit. Therefore, the syringe is inexpensive.

Other objectives, advantages and features of the present invention will be apparent from the following description referring to the attached drawings.

### BRIEF DESCRIPTION OF DRAWINGS

The present invention will be described via detailed illustration of the preferred embodiment referring to the drawings wherein:

FIG. 1 is an exploded view of a push-and-separate safety syringe according to the preferred embodiment of the present invention;

FIG. 2 is an enlarged, partial, cross-sectional view of the push-and-separate safety syringe shown in FIG. 1;

FIG. 3 is an enlarged, partial, cross-sectional view of the push-and-separate syringe shown in FIG. 1 after assembly;

FIG. 4 is a cross-sectional view of the push-and-separate safety syringe shown in FIG. 1 after assembly;

FIG. 5 is another cross-sectional view of the push-and-separate safety syringe in another position than shown in FIG. 4; and

FIG. 6 is another cross-sectional view of the push-and-separate safety syringe in another position than shown in FIG. 5.

FIG. 7 is a cross-sectional view of the conventional syringe.

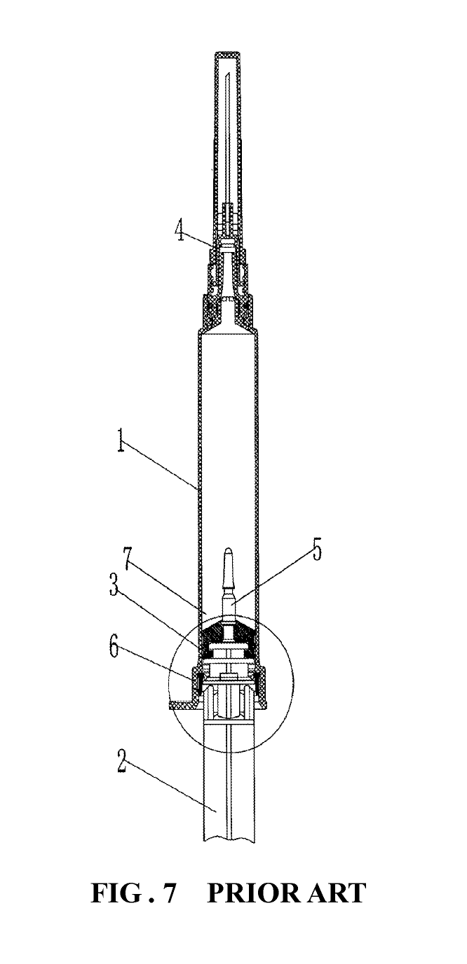

### DETAILED DESCRIPTION OF PREFERRED EMBODIMENT

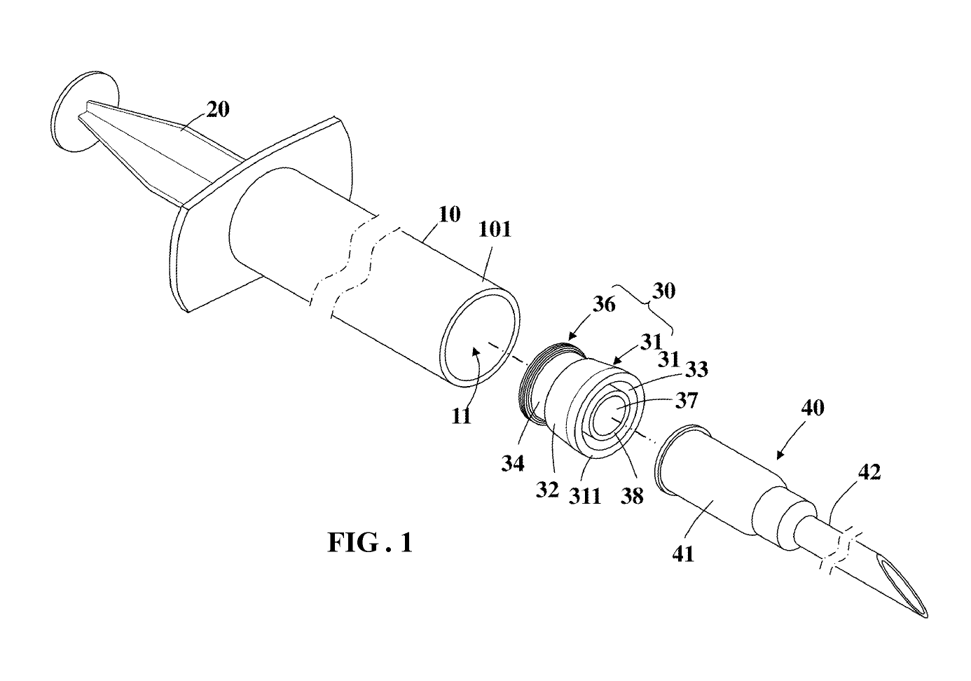

Referring to FIG. 1, a push-and-separate safety syringe includes a barrel 10, a plunger 20 movably inserted in the barrel 10, a connector 30 connected to a front end 101 of the barrel 10, and a needle unit 40 connected to a front end of the connector 30 according to the preferred embodiment of the present invention.

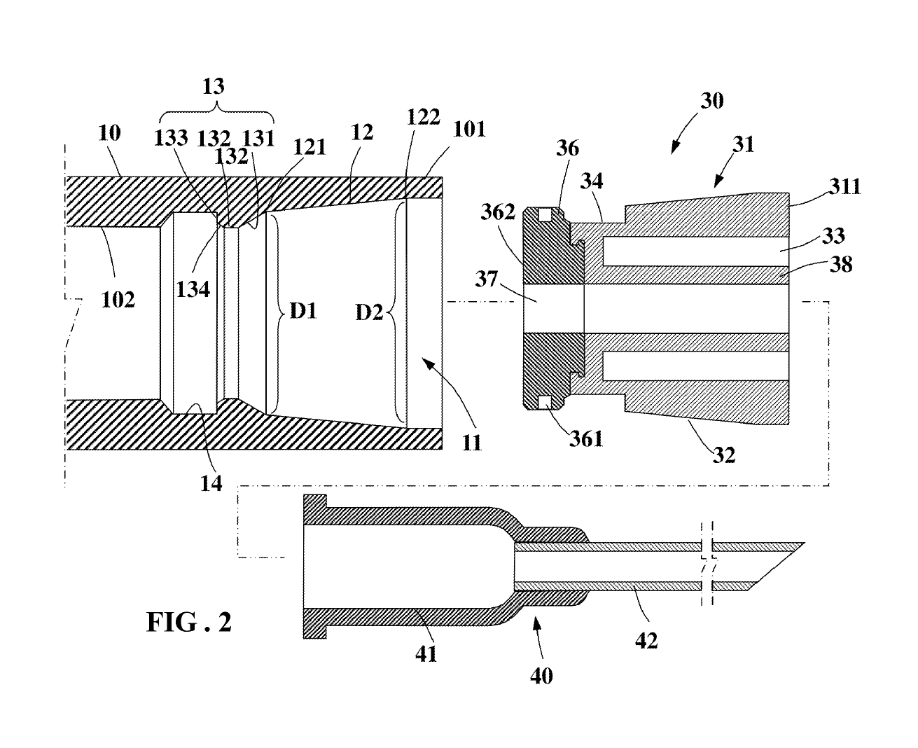

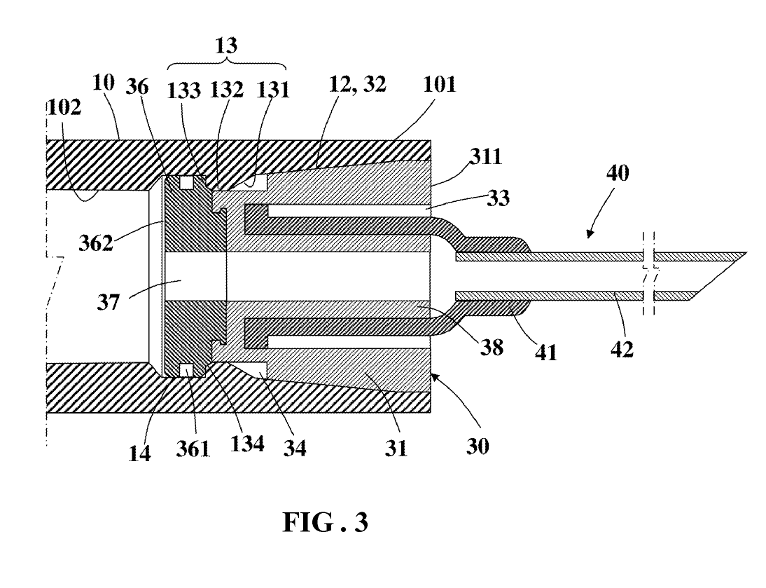

Referring to FIGS. 1 to 3, the connector 30 is inserted in a socket 11 formed at the front end 101 of the barrel 10. The socket 11 includes a conical internal face 12 formed at the front end 101 of the barrel 10, an annular internal rib 13 formed next to the conical internal face 12, and an annular internal groove 14 made next to the annular internal rib 13. The conical internal face 12 is made with a smaller diameter D1 at a rear end 121 and a larger diameter D2 at a front end 122. The annular internal rib 13 includes an annular front face 131, an annular middle face 132, and an annular rear face 133. The annular middle face 132 is formed between the annular front face 131 and the annular rear face 133. The annular front face 131 is tapered as it extends from the rear end 121 of the conical internal face 12. The annular middle face 132 is connected to a smaller end of the annular front face 131. The annular middle face 132 extends parallel to an internal side 102 of the barrel 10. The annular rear face 133 extends perpendicular to the internal side 102 of the barrel 10. There is a chamfer 134 between the annular rear face 133 and the annular middle face 132. The annular internal groove 14 is made in the internal side 102 of the barrel 10.

The connector 30 includes a joint 31, an anchoring ring 36 connected to a rear end of the joint 31, and an axial channel 37 made throughout the joint 31 and the anchoring ring 36. The joint 31 includes a conical external face 32 in compliance with the conical internal face 12 of the barrel 10. The joint 31 includes an annular cavity 33 made around an axial channel 37. The annular cavity 33 includes an open end at a front end 311 of the joint 31. Thus, a tubular insert 38 is formed between the axial channel 37 and the annular cavity 33. The joint 31 further includes an annular external groove 34 made next to a smaller end of the conical external face 32. The anchoring ring 36 is made of an elastic material such as rubber. An external diameter of the anchoring ring 36 is identical to an internal diameter of the annular internal groove 14 of the barrel 10. An annular vent 361 is made in an external side of the anchoring ring 36. The anchoring ring 36 includes a flat rear end 362.

The connector 30 is inserted in the socket 11, which is formed at the front end 101 of the barrel 10. The conical external face 32 is in tight contact with the conical internal face 12. The front end 311 of the joint 31 is flush with the front end 101 of the barrel 10. The annular internal rib 13 of the socket 11 is inserted in the annular external groove 34 of the connector 30. The anchoring ring 36 of the connector 30 is inserted in the annular internal groove 14 of the socket 11.

During the insertion of the connector 30 into the socket 11, the annular front face 131 of the annular internal rib 13 presses and guides the anchoring ring 36 into the annular internal groove 14 via the annular front face 131 and annular middle face 132. The external side of the anchoring ring 36 is in tight contact with an internal side of the annular internal groove 14 to achieve air-tightness. The annular rear face 133 of the annular internal rib 13 stops a front end of the anchoring ring 36 so that the connector 30 cannot be easily detached from the barrel 10 in an opposite direction. Furthermore, the annular internal rib 13 is inserted in the annular external groove 34 of the connector 30 to further restrain the connector 30. The conical internal face 12 of the socket 11 is in compliance with the conical external face 32 of the connector 30 so that they are in tighter contact with each other as the connector 30 is further inserted into the socket 11 to render excellent the air-tightness between the connector 30 and the socket 11.

A piston 21 is connected to a front end of the plunger 20. The piston 21 is made of an elastic material. The piston 21 includes a conical block 22 axially formed with at a front end. The needle unit 40 includes a needle seat 41 and a needle 42 inserted in and connected to a front end of the needle seat 41. The plunger 20 and the needle unit 40 are made in a conventional manner. That is, a conventional plunger and a conventional needle unit can be used in the push-and-separate safety syringe of the present invention. Hence, the cost of the push-and-separate safety syringe of the present invention is low.

The needle seat 41 of the needle unit 40 is inserted in the annular cavity 33 while the tubular insert 38 is inserted in the needle seat 41 of the needle unit 40. The needle 42 and the needle seat 41 are covered by a plastic cap (not shown) before the needle unit 40 is used. A medical worker will not touch and be hurt by the needle 42 while handing the needle unit 40. Sometimes, the medical worker twists the needle seat 41 while handling the needle unit 40. Thus, the tubular insert 38 is inserted deeply in the needle seat 41. The compliance of the conical internal face 12 with the conical external face 32 enables the connector 30 to stand the twist of the needle seat 41, without moving further into the barrel 10.

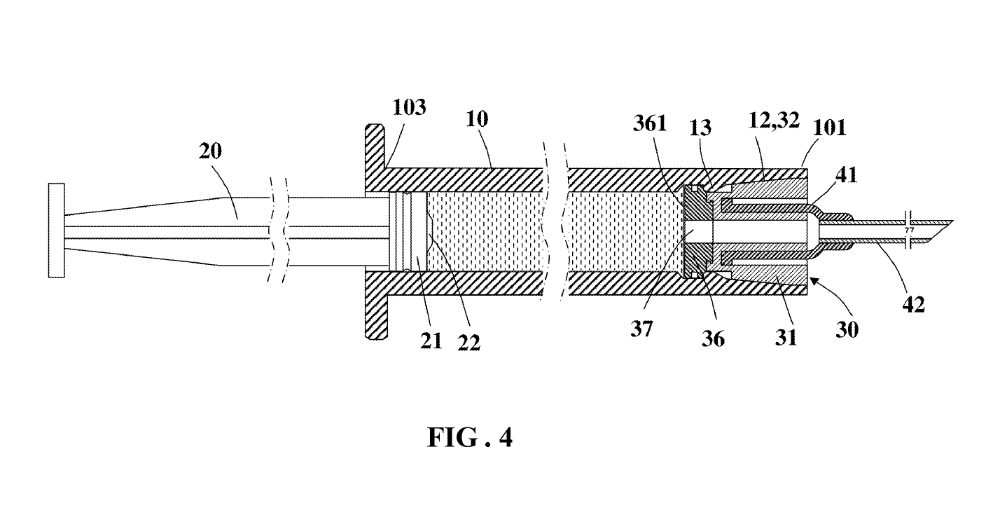

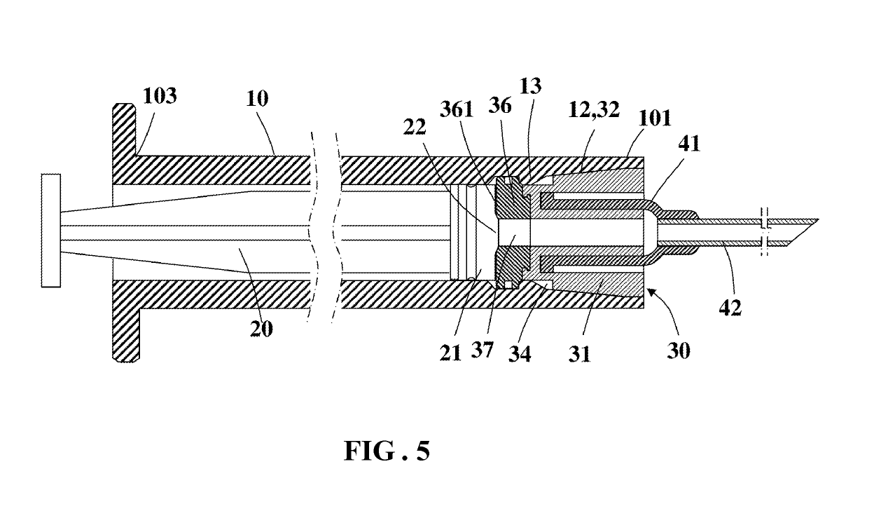

Referring to FIGS. 4 and 5, the plunger 20 can be freely reciprocated in the barrel 10. The plunger 20 can be moved forward from the rear end 103 of the barrel 10 so that the piston 21 attached to the front end of the plunger 20 is in contact with the rear end 362 of the anchoring ring 36 of the connector 30. The forward movement of the plunger 20 expels air out of the barrel 10 via the needle 42 when the barrel 10 is clear of medicine. It is necessary to expel the air out of the barrel 10 before medicine is drawn into the barrel 10. Then, the needle 42 is inserted in a bottle filled with medicine, and the plunger 20 is moved backward to draw some of the medicine into the barrel 10 via the needle 42. It should be noted that the connector 30 and the needle unit 40 are not interfered with by the forward movement of the plunger 20 to expel the air or the backward movement of the plunger 20 to draw the medicine. Then, the medical worker uses the syringe to inject the medicine into a patient. The medical worker moves the plunger 20 forward to inject the medicine into the patient from the barrel 10 via the needle 42. It should be noted that the compliance of the conical internal face 12 with the conical external face 32 keeps the connector 30 and the needle 42 stable without shaking when the needle 42 is inserted into the patient's skin and muscles under resistance. Moreover, the connector 30 is inserted in the front end 101 of the barrel 10 without leaving any portion out of the barrel 10 to interfere with the medical worker's handling of the syringe.

Referring to FIG. 5, the plunger 20 is moved forward to inject the medicine into the patient via the needle 42. The rear end 362 of the anchoring ring 36 of the connector 30 is flat, and the axial channel 37 is axially made in the connector 30. This configuration cooperates with the piston 21 of the plunger 20 so that resistance against the injection of the medicine gets smaller as the piston 21 gets closer to the anchoring ring 36 and the medicine becomes less in the barrel 10. The conical block 22 of the piston 21 enters and hence blocks the axial channel 37 as the front end of the piston 21 is in contact with the rear end 362 of the anchoring ring 36. Hence, not any of the medicine is left between the piston 21 and the anchoring ring 36. Some of the medicine (the “residual medicine”) is inevitably left in the axial channel 37 and the needle 42. However, the amount of the residual medicine is limited and can be ignored. An adequate dose of the medicine is given to the patient. In fact, the residual medicine prevents air from entering the patient via the needle 42 that would otherwise cause a blood clot in the patient.

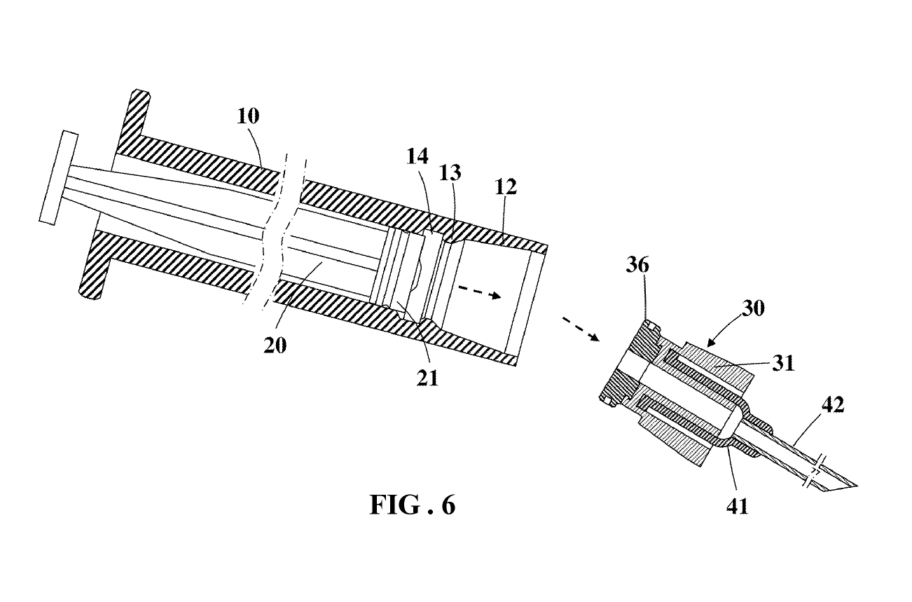

Referring to FIG. 6, after the injection, the medical worker removes the needle 42 from the patient's skin and muscles under little resistance. In addition, the connector 30 is firmly inserted in the barrel 10. Hence, the connector 30 is not loosened in the barrel 10 during the detachment of the needle 42 from the patient's skin and muscles. To detach the needle unit 40 from the barrel 10, the medical worker pushes the plunger 20 further forward so that the rear end 362 of the anchoring ring 36 is moved forward by the piston 21. Almost all of the forward force is transferred to the rear end 362, which is flat, from the piston 21. The anchoring ring 36 pushes the annular rear face 133 of the annular internal rib 13 forward because the annular rear face 133 includes the chamfer 134, which facilitates slight deformation of the anchoring ring 36 under squeeze. Thus, the anchoring ring 36 is allowed to move past the annular middle face 132 of the annular internal rib 13. Moreover, the annular vent 361 of the anchoring ring 36 facilitates the deformation of the anchoring ring 36 under squeeze and the movement of the anchoring ring 36 past the annular internal rib 13. When the anchoring ring 36 completely leaves the annular internal rib 13, the tight contact of the conical external face 32 with conical internal face 12 is terminated and the connector 30 and the needle unit 40 are detached from the barrel 10. It should be noted that the detachment of the needle unit 40 from the barrel 10 is done with a single hand, like the injection of the medicine, only marginally harder. The ability to use of a single hand to detach the needle unit 40 from the barrel 10 is important because the medical worker has to use the other hand to press a sterilized cotton ball soaked with alcohol against the patient. The medical worker can use a single hand to aim the needle unit 40 at a trash can and push the plunger 20 to cast the needle unit 40 into the trash can from the barrel 10. The period in which the needle unit 40 is exposed is limited.

As discussed above, all of the problems with the self-locking self-destroying syringe mentioned in the RELATED PRIOR ART are overcome by the safety syringe of the present invention.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

1. A push-and-separate safety syringe comprising:
a barrel 10;
a socket 11 formed in the barrel, comprising
a conical internal face 12 formed next to a front end of the barrel 10 and comprising a rear end 121 and a front end 122 made with a diameter larger than that of the rear end 121;
an annular internal rib 13 formed at a rear end 121 of the conical internal face 12; and
an annular internal groove 14 made next to the annular internal rib 13;
a plunger 20 movably inserted in the barrel 10;
a connector comprising:
a joint 31 comprising a conical external face 32 in compliant contact with the conical internal face 12;
an annular cavity 33 made in the joint 31;
an anchoring ring 36 formed at a rear end of the joint 31 and inserted in the annular internal groove 14;
an axial channel 37 made throughout the joint 31 and the anchoring ring 36;
a tubular inset 38 formed between the annular cavity 33 and the axial channel 37; and
an annular external groove 34 made between the joint 31 and the anchoring ring 36 to receive the annular internal rib 13; and
a needle unit 40 comprising:
a needle seat 41 inserted in the annular cavity 33 while the tubular insert 38 is fitted in the needle seat 41; and
a needle 42 connected to a front end of the needle seat 41.

2. The push-and-separate safety syringe according to claim 1, wherein the annular internal rib 13 is inserted in the annular external groove 34.

3. The push-and-separate safety syringe according to claim 2, wherein the annular internal rib 13 comprises:
an annular front face 131 being tapered and extending from a rear end 121 of the conical internal face 12, to facilitate sliding of the anchoring ring 36 on the annular front face 131 during the insertion of the connector 30 into the socket 11;
an annular rear face 133 extending perpendicular to an internal side 102 of the barrel 10 to abut against the anchoring ring 36 to keep the anchoring ring 36 in the annular internal groove 14; and
an annular middle face 132 formed between the annular front face 131 and the annular rear face 133.

4. The push-and-separate safety syringe according to claim 3, wherein the socket 11 further comprises a chamfer 134 formed between the annular rear face 133 and the annular middle face 132.

5. The push-and-separate safety syringe according to claim 1, wherein the rear end 362 of the anchoring ring 36 is a flat end.

6. The push-and-separate safety syringe according to claim 1, wherein the anchoring ring 36 comprises an annular vent 37 made in a periphery thereof.
